(19) United States
(12) Patent Application Publication
(10) Pub. No.: US 2026/0191423 A1
(43) Pub. Date: Jul. 9, 2026

(54) SYSTEM AND METHOD FOR CARDIOVASCULAR RISK MONITORING

(71) Applicant: Sharad Chandra Jaitly, Greenwich, CT (US)

(72) Inventor: Sharad Chandra Jaitly, Greenwich, CT (US)

(21) Appl. No.: 19/557,022

(22) Filed: Mar. 4, 2026

Related U.S. Application Data
(63) Continuation-in-part of application No. 18/365,496, filed on Aug. 4, 2023, now abandoned.

(51) Int. Cl. A61B 5/024; A61B 5/00

(52) U.S. Cl. CPC A61B 5/02405; A61B 5/02438; A61B 5/6826; A61B 5/7275; A61B 5/742; A61B 5/7465

(57) ABSTRACT

A system and method for monitoring cardiovascular risk comprises measuring heart rate variability (HRV) using a dedicated HRV measurement device. The HRV measurement device determines a cardiovascular risk score from an average HRV measurement of a patient and indicates risk status through a visual display. The system also transmits the HRV measurements and cardiovascular risk scores to the mobile devices of the patient and their healthcare provider, providing an early alert of the risk of sudden cardiac arrest.

### CROSS-REFERENCE TO RELATED APPLICATIONS

This application is a continuation-in-part of U.S. Non-Provisional application Ser. No. 18/365,496, filed Aug. 4, 2023, which is hereby incorporated by reference, to the extent that it is not conflicting with the present application.

### BACKGROUND OF INVENTION

### 1. Field of the Invention

The invention relates generally to systems and methods of cardiovascular risk measurement, and more particularly to the use of heart rate variability measurements to monitor cardiovascular risk.

### 2. Description of the Related Art

Up to 20% of cardiac deaths are sudden, without preceding symptoms. In the U.S. alone, 356,000 deaths are recorded annually from sudden cardiac arrests/deaths (SCA/SCD), which translates to an average of 900-1000 deaths occurring daily. Worldwide, the figures may be up to 17 million deaths annually. SCDs occur mostly without warning and often in younger to middle-aged populations. However, there may be one or more cardiac risk factors that, if detected earlier, could provide an early warning that could save thousands of lives.

Heart rate variability (HRV) is increasingly being recognized as a cardiac risk marker, but to date has not been used frequently by healthcare professionals due to a lack of specific devices and protocols. HRV is the physiological variation that exists between the sinus rhythm of the heart beats. There is a sympathetic set of (accelerator) nerve fibers and there is a parasympathetic set (decelerator) set as well, actively sending signals to the heart continuously. Together these sets of nerve fibers form the autonomic nervous system (ANS) The balance between these two sets of fibers along with the physiological respiratory cycles of inspiration & expiration determines the HRV in that instant for that individual.

The normal range of HRV has been found to be anywhere between 50-90 milliseconds at the resting phase of an individual. If HRV averages less than 50 msec at resting phase in several readings at various times of the day, this signifies a lack of variability or lack of adaptability of the heart to change its rate in response to fright, flight, freeze or other stress situations implying a pathological response or in readiness to cardiac stress situations. It follows that the greater the variability, the greater the adaptability of the heart to react to a stress situation. A low HRV of less than 50 msec may be seen or recognized as a cardiac risk marker found commonly in diabetes, metabolic syndromes, cardiac genetic disorders, heart failure, serious cardiac arrhythmias, coronary artery disease, or other forms of heart diseases.

Therefore, there is a need to solve the problems described above by providing a device and method dedicated to monitoring and determining the risk of SCA/SCD based on HRV measurements.

The aspects or the problems and the associated solutions presented in this section could be or could have been pursued; they are not necessarily approaches that have been previously conceived or pursued. Therefore, unless otherwise indicated, it should not be assumed that any of the approaches presented in this section qualify as prior art merely by virtue of their presence in this section of the application.

### BRIEF INVENTION SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In an aspect, an HRV measurement device provides an indication of whether a measurement is normal, borderline, or abnormal, thus providing the advantage of an early indication of an abnormal cardiovascular risk factor.

In another aspect, an HRV measurement device is dedicated to HRV measurement, providing the advantage of measurements that are easy to interpret and not overshadowed by a multitude of other biomarker measurements and device features.

In another aspect, a method of cardiovascular risk monitoring provides real-time transmission of HRV data and risk status to a healthcare provider, thus providing the advantage of early warning of potential cardiovascular health problems that might otherwise go undetected.

The above aspects or examples and advantages, as well as other aspects or examples and advantages, will become apparent from the ensuing description and accompanying drawings.

### BRIEF DESCRIPTION OF THE DRAWINGS

For exemplification purposes, and not for limitation purposes, aspects, embodiments or examples of the invention are illustrated in the figures of the accompanying drawings, in which:

FIG. 1 illustrates a top perspective view of the interior of an HRV measurement device, according to an aspect.

FIGS. 2A-2B illustrate a top perspective view and a bottom perspective view, respectively, of the exterior of an HRV measurement device, according to an aspect.

FIGS. 3A-3F illustrates a top view, top perspective view, left side view, front view, bottom view, and bottom perspective view, respectively, of a housing of an HRV measurement device, according to an aspect.

FIG. 4 illustrates an exploded view of a housing of an HRV measurement device, according to an aspect.

FIGS. 5A-5F illustrate a right side view, rear view, top view, front view, left side view, and bottom view, respectively, of a cover of an HRV measurement device, according to an aspect.

FIGS. 6A-6E illustrate a right side view, top view, front view, left side view, and bottom view, respectively, of a housing lid of an HRV measurement device, according to an aspect.

FIGS. 7A-7F illustrate a right side view, rear view, top view, front view, left side view, and bottom view, respectively, of a housing body of an HRV measurement device, according to an aspect.

FIG. 8 illustrates internal components of an HRV measurement device, according to an aspect.

FIG. 9 illustrates a system for monitoring cardiovascular risk using HRV measurements, according to an aspect.

FIG. 10 illustrates a method of determining an average HRV, according to an aspect.

FIGS. 11A-11B illustrate examples of user interfaces for a patient and a healthcare provider, respectively, according to an aspect.

### DETAILED DESCRIPTION

What follows is a description of various aspects, embodiments and/or examples in which the invention may be practiced. Reference will be made to the attached drawings, and the information included in the drawings is part of this detailed description. The aspects, embodiments and/or examples described herein are presented for exemplification purposes, and not for limitation purposes. It should be understood that structural and/or logical modifications could be made by someone of ordinary skills in the art without departing from the scope of the invention. Therefore, the scope of the invention is defined by the accompanying claims and their equivalents.

It should be understood that, for clarity of the drawings and of the specification, some or all details about some structural components or steps that are known in the art are not shown or described if they are not necessary for the invention to be understood by one of ordinary skills in the art.

As used herein and throughout this disclosure, the term “mobile device” refers to any electronic device capable of communicating across a mobile network. A mobile device may have a processor, a memory, a transceiver, an input, and an output. Examples of such devices include cellular telephones, personal digital assistants (PDAs), portable computers, etc. The memory stores applications, software, or logic. Examples of processors are computer processors (processing units), microprocessors, digital signal processors, controllers and microcontrollers, etc. Examples of device memories that may comprise logic include RAM (random access memory), flash memories, ROMS (read-only memories), EPROMS (erasable programmable read-only memories), and EEPROMS (electrically erasable programmable read-only memories). A transceiver includes but is not limited to cellular, GPRS, Bluetooth, and Wi-Fi transceivers.

“Logic” as used herein and throughout this disclosure, refers to any information having the form of instruction signals and/or data that may be applied to direct the operation of a processor. Logic may be formed from signals stored in a device memory. Software is one example of such logic. Logic may also be comprised by digital and/or analog hardware circuits, for example, hardware circuits comprising logical AND, OR, XOR, NAND, NOR, and other logical operations. Logic may be formed from combinations of software and hardware. On a network, logic may be programmed on a server, or a complex of servers. A particular logic unit is not limited to a single logical location on the network.

Mobile devices communicate with each other and with other elements via a network, for instance, a cellular network. A “network” can include broadband wide-area networks, local-area networks, and personal area networks. Communication across a network can be packet-based or use radio and frequency/amplitude modulations using appropriate analog-digital-analog converters and other elements. Examples of radio networks include GSM, CDMA, Wi-Fi and BLUETOOTH® networks, with communication being enabled by transceivers. A network typically includes a plurality of elements such as servers that host logic for performing tasks on the network. Servers may be placed at several logical points on the network. Servers may further be in communication with databases and can enable communication devices to access the contents of a database. For instance, an authentication server hosts or is in communication with a database having authentication information for users of a mobile network. A “user account” may include several attributes for a particular user, including a unique identifier of the mobile device(s) owned by the user, relationships with other users, call data records, bank account information, etc. A billing server may host a user account for the user to which value is added or removed based on the user's usage of services. One of these services includes mobile payment. In exemplary mobile payment systems, a user account hosted at a billing server is debited or credited based upon transactions performed by a user using their mobile device as a payment method.

For the following description, it can be assumed that most correspondingly labeled elements across the figures (e.g., 106 and 206, etc.) possess the same characteristics and are subject to the same structure and function. If there is a difference between correspondingly labeled elements that is not pointed out, and this difference results in a non-corresponding structure or function of an element for a particular embodiment, example or aspect, then the conflicting description given for that particular embodiment, example or aspect shall govern.

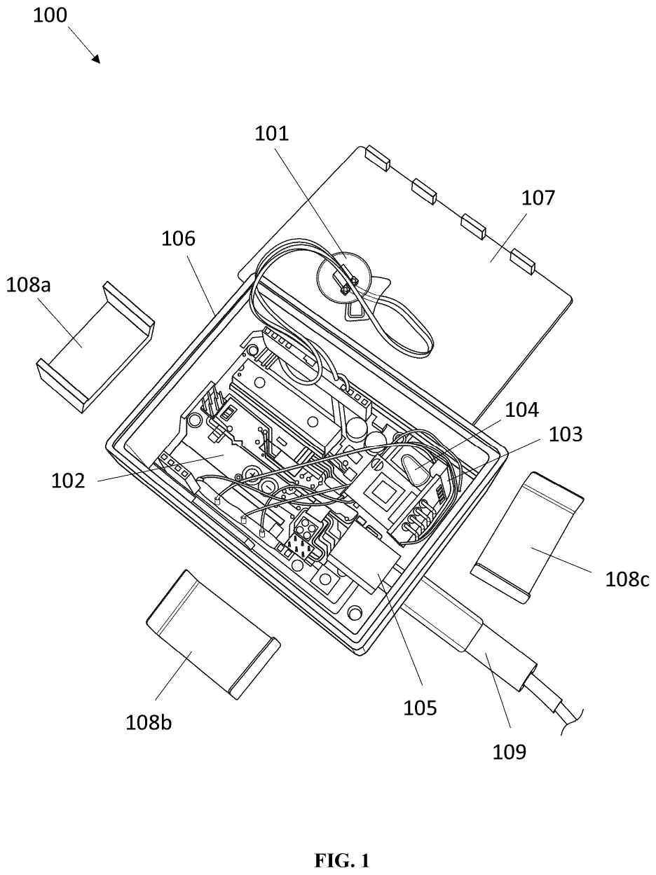

FIG. 1 illustrates a top perspective view of the interior of a heart rate variability (HRV) measurement device 100, according to an aspect. In an embodiment, the HRV measurement device comprises a pulse sensor 101, microcontroller 102 (e.g., Arduino UNO Rev3; Adafruit Feather MO Adalogger), RGB LED Module 103 with LED 104, and USB port 105. The microcontroller 102 and LED module 103 are protected within a housing body 106, the pulse sensor 101 is disposed on a housing lid 107, and the housing lid 107 may comprise a clear window (not shown) for viewing the LED 104. When the housing lid 107 is closed onto the housing body 106, clips 108a-108c may be used to secure the lid 107 to the housing body 106. A USB cable 109 may be connected to the USB port 105 to deliver power from a power source (e.g., a laptop computer). In an embodiment, the HRV measurement device 100 may be configured to receive power through a power jack using an AC-to-DC adapter, or it may be powered by a battery or using solar energy. In an embodiment, the HRV measurement device 100 may also comprise a wireless communication module (e.g., Wi-Fi, Bluetooth, etc.) and a memory card reader.

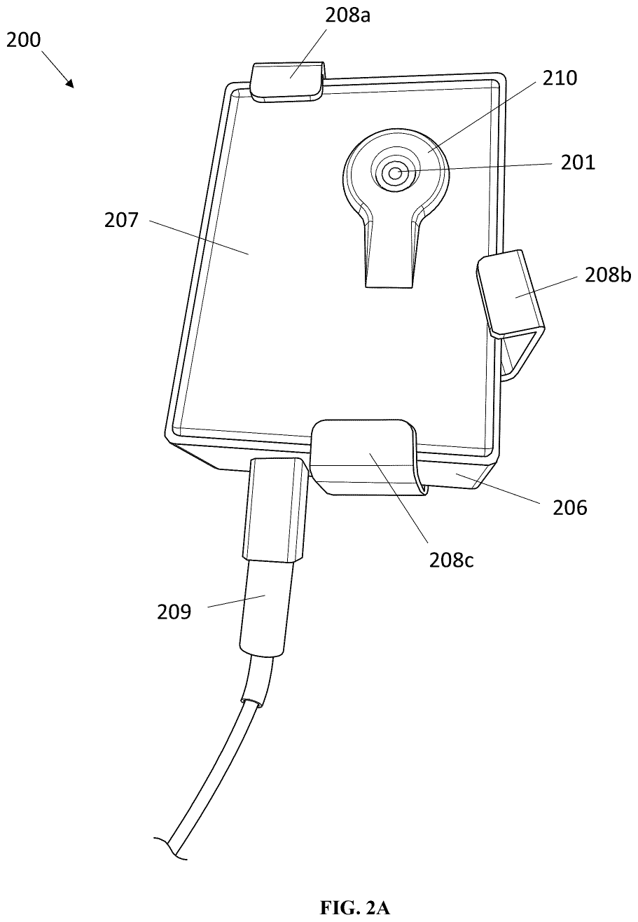

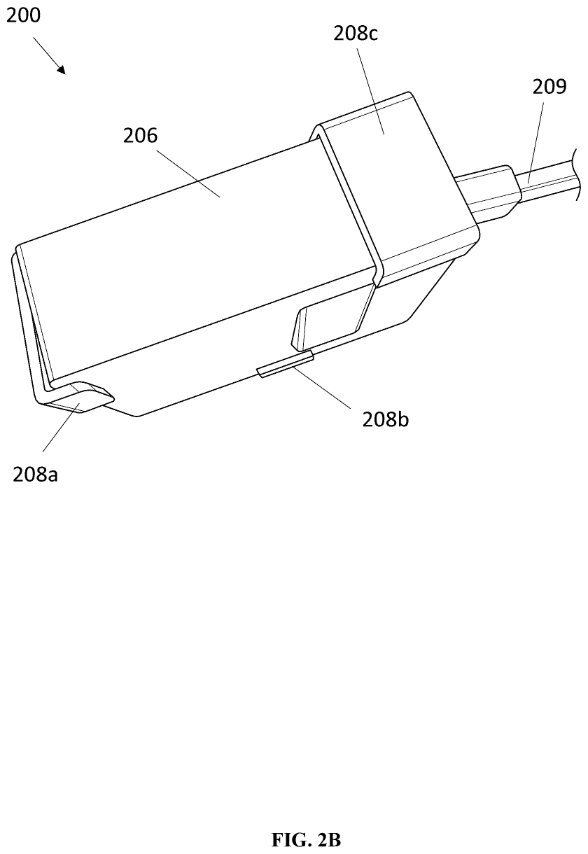

FIGS. 2A-2B illustrate a top perspective view and a bottom perspective view, respectively, of the exterior of an HRV measurement device 200, according to an aspect. In an embodiment, the HRV measurement device 200 comprises a housing body 206 and a housing lid 207 secured together using clips 208a-208c. In alternative embodiments, fewer than 3 clips or more than 3 clips may be used. The housing lid 207 includes an opening for passage of light to and from a pulse sensor 201. A USB cable 209 may be used to provide power to the HRV measurement device 200 from a power source. In an embodiment, the housing lid 207 may also include a conformable finger pad 210 to cushion a user's finger when placed against the pulse sensor 201. In an example, the housing body 206 may be 3.50″×2.75″×1.33″ in size and constructed of lightweight but durable materials such as thermoplastics (e.g., Prusament PETG). The housing lid 207 may be constructed of the same or different materials as the housing body 206. In alternative embodiments, the housing body 206 and housing lid 207 may be secured together by screws or other fasteners.

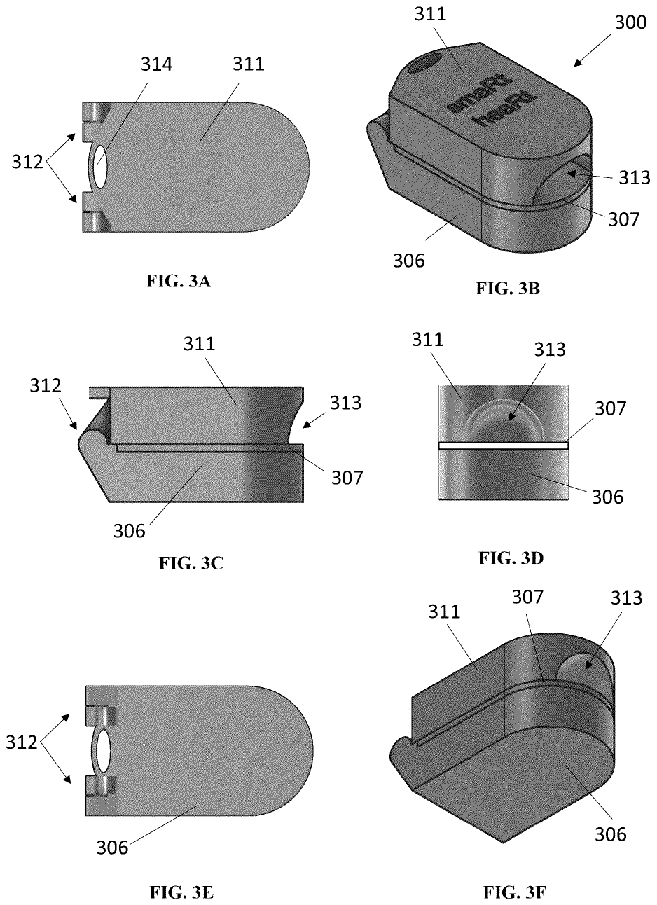

FIGS. 3A-3F illustrate a top view, top perspective view, side view, front view, bottom view, and bottom perspective view, respectively, of a HRV measurement device 300, according to an aspect. In an embodiment, the HRV measurement device comprises a small housing (e.g., 35 mm×60 mm×40 mm) with a housing body 306, housing lid 307, and a cover 311. The cover 311 is pivotally secured to the housing body 306 by a hinge mechanism 312 such that the cover 311 can be rotated from a closed position over the housing lid 307 to an open position exposing the housing lid 307 and a pulse sensor (not shown). In an embodiment, a cover cavity 313 accommodates fingertip placement on the pulse sensor in the housing lid 307, and the cover cavity 313 may also facilitate opening of the cover 311. The cover 311 may include an opening 314 for attachment of a lanyard.

FIG. 4 illustrates an exploded view of a housing of a heart rate variability measurement device, according to an aspect. In an embodiment, the housing comprises a housing body 406, a housing lid 407, and a cover 411. In an embodiment, the cover 411 comprises a cover cavity 413 to accommodate a fingertip and an opening 414 for attachment of a lanyard or other accessory. The housing lid 407 may comprise an opening 415 for a pulse sensor (see below) which a user will contact with their finger. The housing lid 407 may be secured to the housing body 406 by screws (not shown) or other means that allow removal for changing a battery or for other service needs. The cover 411 is pivotally attached to the housing body 406 through a hinge mechanism. In an embodiment, the hinge mechanism comprises housing body posts 416a, 416b projecting from housing body arms 417a, 417b located on a rear wall of housing body 406, wherein holes in cover arms (cover hole 418b and cover arm 419b are visible in FIG. 4) receive the housing body posts 416a, 416b such that the cover 411 is secured to the housing body 406 but may rotate about the housing body posts 416a, 416b to allow the cover to be opened and closed over the housing lid 407. The housing body 406 may comprise a housing body cavity 420 configured to hold the internal components of the heart rate variability device.

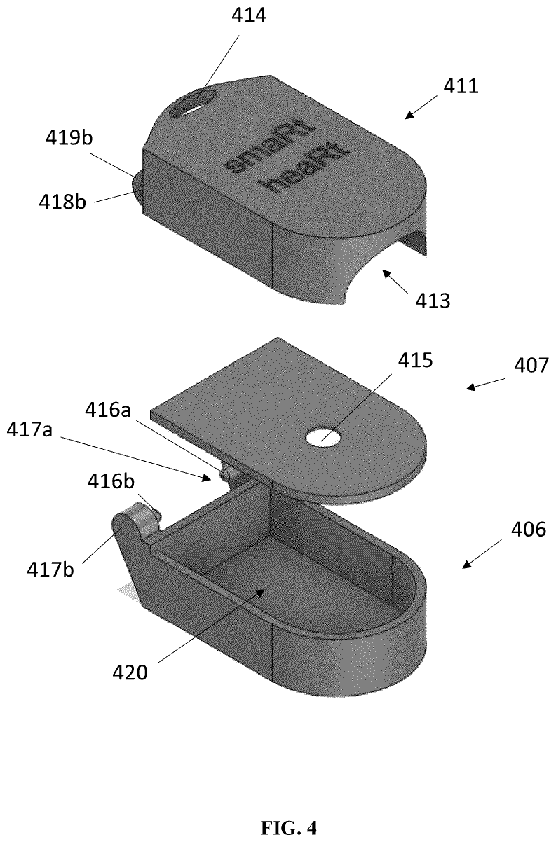

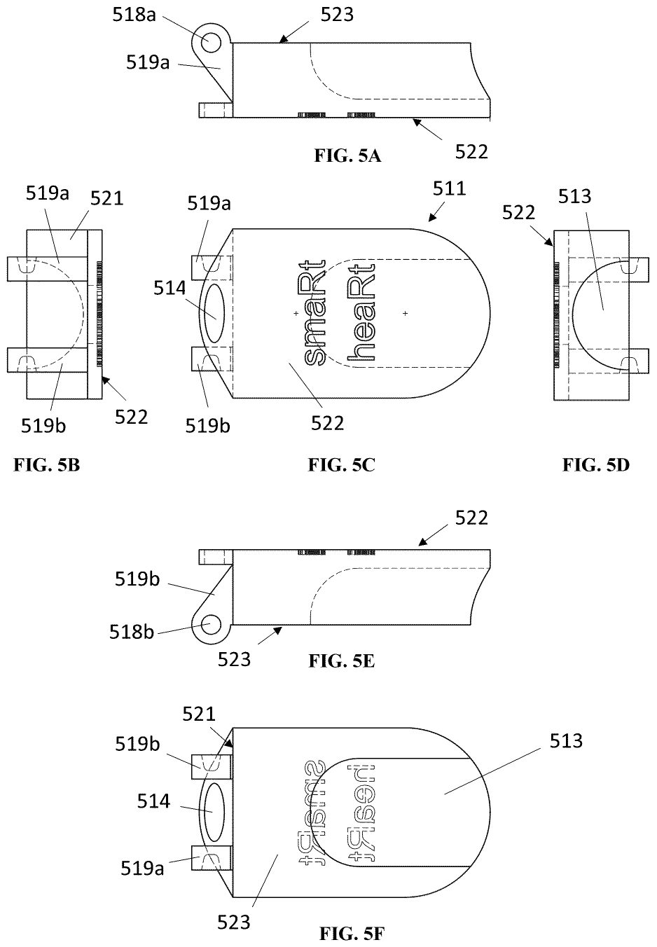

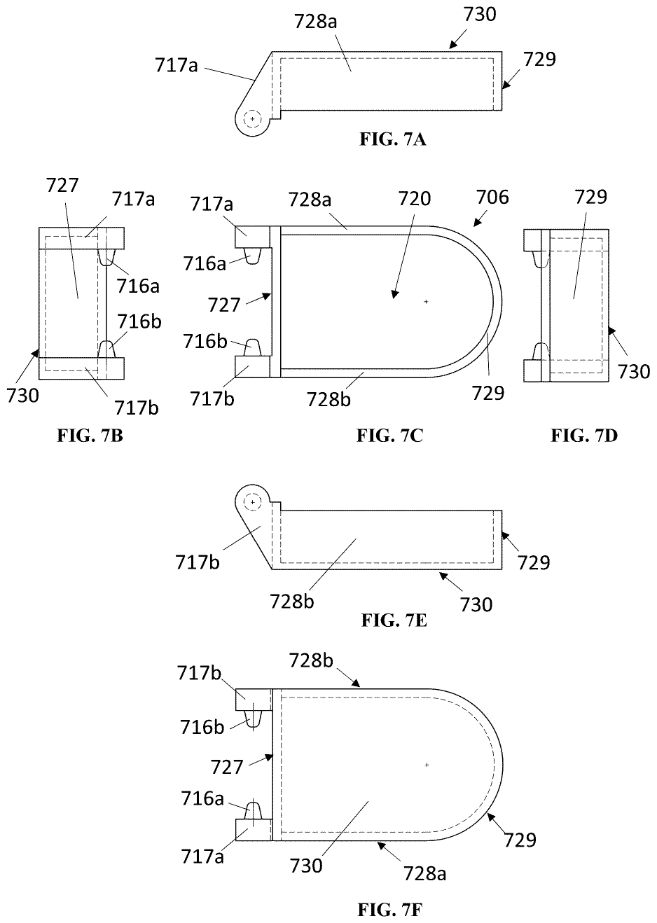

FIGS. 5A-5F illustrate a right side view, rear view, top view, front view, left side view, and bottom view, respectively, of a cover 511 of a heart rate variability measurement device, according to an aspect. In an embodiment, the cover 511 comprises cover holes 518a, 518b in cover arms 519a, 519b extending from a cover rear surface 521, wherein the cover holes 518a, 518b are configured to receive housing body posts (e.g., housing body posts 716a, 716b described below and in FIG. 7) to form a hinge mechanism. In an embodiment, a cover top surface 522 may comprise an opening 514 for attachment of a lanyard or other accessory. A cover bottom surface 523 may comprise a rounded cover cavity 513 to accommodate a fingertip during HRV measurement.

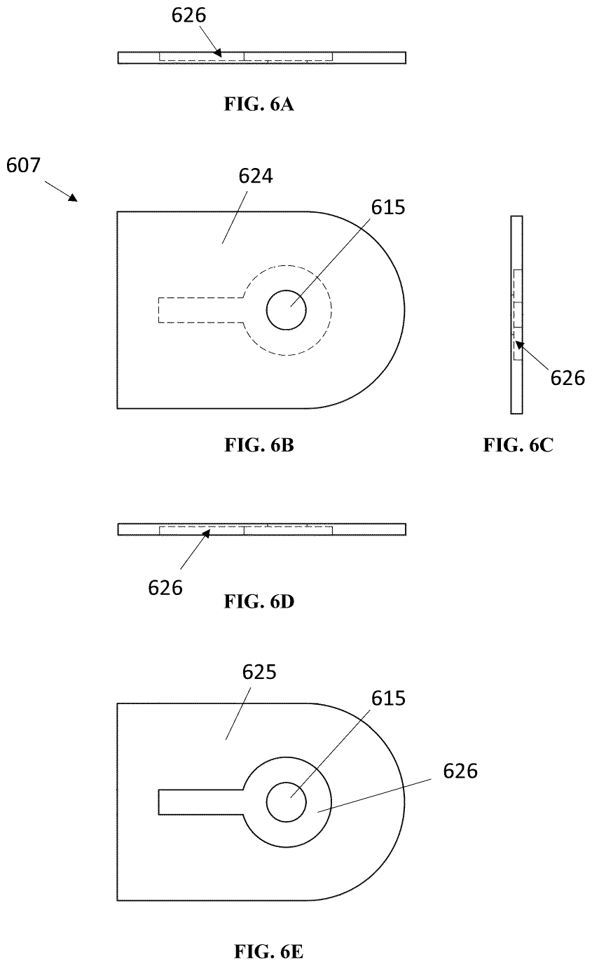

FIGS. 6A-6E illustrate a right side view, top view, front view, left side view, and bottom view, respectively, of a housing lid 607 of a heart rate variability measurement device, according to an aspect. In an embodiment, the housing lid 607 may comprise a hole 615 that exposes an LED light source and photodetector of a pulse sensor to a fingertip of a user when the fingertip is pressed against a top surface 624 of the housing lid 607. The bottom surface 625 of the housing lid 607 may also comprise a cavity 626 to accommodate and secure the electronics and other components of the pulse sensor to the bottom surface 625 of the housing lid 607. The housing lid 607 may also comprise a clear window for viewing LED indicators (not shown).

FIGS. 7A-7F illustrate a right side view, rear view, top view, front view, left side view, and bottom view, respectively, of a housing body 706 of a heart rate variability measurement device, according to an aspect. In an embodiment, the housing body 706 may comprise a housing body rear wall 727, housing body right side wall 728a, housing body left side wall 728b, housing body front wall 729, and housing body bottom wall 730 that form a cavity 720 configured to hold internal components of a heart rate variability measurement device. The housing body 706 may also comprise housing body posts 716a, 716b extending from housing body arms 717a, 717b located on the housing body rear wall 727, wherein the housing body posts 716a, 716b are configured to be received by holes in a cover (e.g., cover holes 518a, 518b of cover 511 described above and in FIG. 5) to form a hinge mechanism.

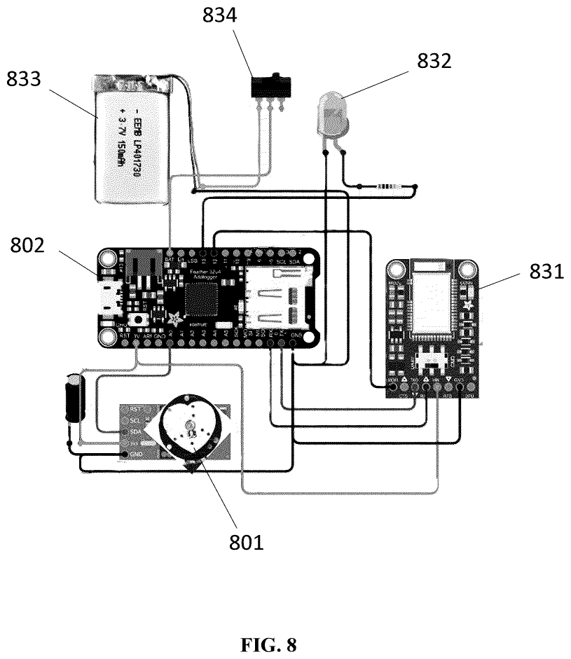

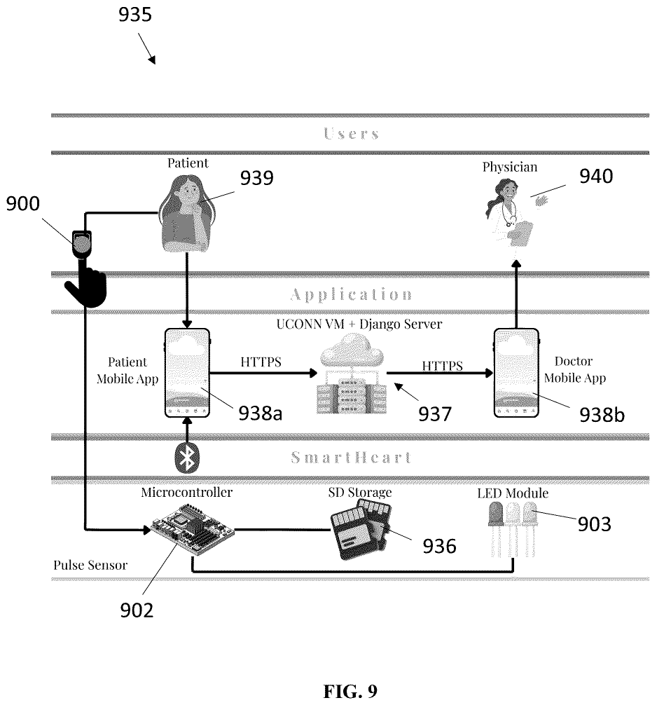

FIG. 8 illustrates internal components of a heart rate variability measurement device, according to an aspect. In an embodiment, a heart rate variability measurement device may comprise a pulse sensor 801, a microprocessor 802 (e.g. Arduino UNO Rev3; Adafruit Feather MO Adalogger), a wireless communication module 831 (e.g., Bluetooth), a status LED 832 for indication of device status, a battery 833 for providing power to the device, and a power switch 834 for turning the device on and off. In an example, the status LED 832 may illuminate green and blink slowly when collecting data, yellow when not collecting data or if there is an error, and red when the battery is low.

FIG. 9 illustrates a system 935 for monitoring cardiovascular risk using HRV measurements, according to an aspect. In an embodiment, the cardiovascular risk monitoring system 935 comprises an HRV measurement device 900 comprising a pulse sensor, microcontroller 902, LED module 903, wireless communication module (not shown), and may also be configured to transfer data to removable data storage media such as SD cards 936. The cardiovascular risk monitoring system 935 may also comprise a cloud server 937, and one or more mobile devices 338a, 338b for transmission and viewing of HRV data. In an embodiment, the HRV measurement device 900 may store data from multiple users, wherein the data is uniquely and securely encrypted for retrieval by a physician.

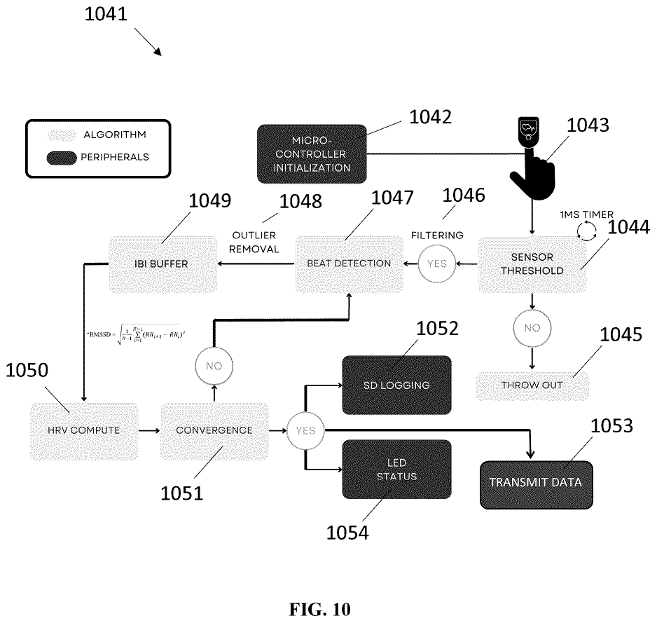

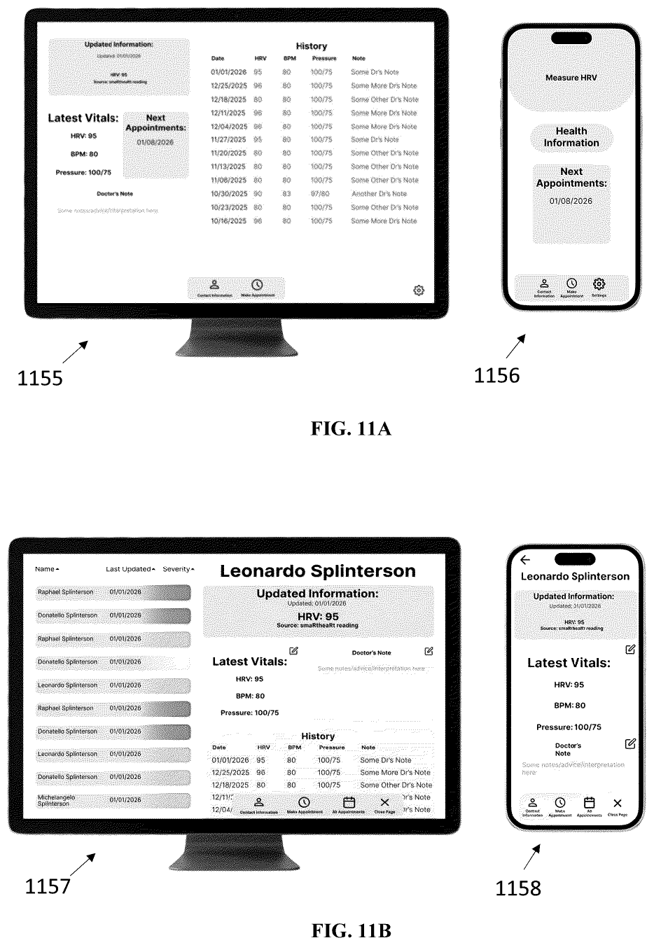

In an embodiment, the cardiovascular risk monitoring system 935 is used in a method of monitoring cardiovascular risk using HRV measurements, according to an aspect. In an embodiment, a first step of a cardiovascular risk monitoring method comprises a patient 939 placing a finger or thumb on a finger pad of a pulse sensor of an HRV measurement device 900 for a predefined duration of time sufficient to determine an HRV measurement (e.g., at least 60 seconds). A second step comprises measurement and determination of an HRV value by the HRV measurement device 900 (see below and FIG. 10 for details). A third step comprises determining a cardiovascular risk score and indicating risk status to the user using an LED light indicator 903 (e.g., green for normal, yellow for borderline, red for abnormal) on the HRV measurement device 900 and/or by display on a patient mobile device 938a. A cardiovascular risk score may be a risk category assigned based on predetermined HRV ranges (e.g., HRV greater than 100 ms is normal, HRV between 51-99 ms is borderline, and HRV <50 ms is abnormal). A fourth step comprises transmission of the HRV data to a healthcare provider 940 for assessment, wherein the data may be uploaded to a cloud server 937 and subsequently transmitted to a healthcare provider mobile device 938b, and the data may also be stored on removable storage media 936 to be provided to the healthcare provider 940. Transmission of the HRV data from the HRV measurement device 900 to the patient mobile device 938a, uploading to the cloud server 937, and transmission to the healthcare provider mobile device 938b may occur automatically and in real-time. Alternatively, data may be transmitted to the healthcare provider cumulatively after a period of use by the patient (e.g., at the end of each month of use). An additional step may comprise determining an average HRV value after several HRV measurements, each measurement taken over the predefined duration of time, measured several times a day (e.g., at least three times a day, such as morning, afternoon, and evening) and several days per week (e.g., five days a week).

FIG. 10 illustrates a method 1041 of determining an HRV value, according to an aspect. In an embodiment, the method 1041 begins with a microcontroller initialization step 1042 as an HRV measurement device is turned on and a patient places a fingertip into the device to begin measurements by a pulse sensor of the device (e.g., pulse sensor readings sampled at 100 Hz). In a sensor threshold step 1044, a processor of the microcontroller determines whether a pulse sensor reading meets a threshold and either moves to the step 1045 of throwing out the reading or moves on to a data filtering step 1046 and beat detection step 1047. Data filtering may comprise high pass filtering (e.g., 0.5 Hz, equivalent to 30 beats per minute) to remove baseline noise and initial shifting, and data filtering may comprise low pass filtering (e.g., 4 Hz, equivalent to 240 beats per minute) to smooth and remove high frequency noise. The method 1041 may then move on to outlier removal 1048 and Inter-Beat-Interval (IBI) determination 1049 based on R-R intervals. In an embodiment, an HRV computing step 1050 comprises the calculation of a Root Mean Square of Successive Differences (RMSSD) between each heartbeat using R-R intervals. In alternative embodiments, other methods may be used to calculate HRV. In a convergence step 1051, the method returns to the beat detection step 1047 if the HRV computation does not converge, otherwise the calculated HRV is recorded to a memory storage device in a data logging step 1052 and/or transmitted wirelessly in a data transmission step 1053, and the status of the recording is indicated by LED illumination 1054.

FIGS. 11A-11B illustrate examples of user interfaces for a patient and a healthcare provider, respectively, according to an aspect. In an embodiment, as seen in FIG. 11A, a patient may view HRV measurement results through a desktop-based patient user interface 1155 or a mobile-based patient user interface 1156 provided by a software application. The patient user interfaces 1155, 1156 may also display other information, including additional biomarker results, appointment information, doctor's notes, and they may also provide functions such as making new appointments and contacting healthcare providers. In an embodiment, the patient user interfaces 1155, 1156 may be used to initiate a measurement by an HRV measurement device. FIG. 11B illustrates an example of a desktop-based healthcare provider user interface 1157 and a mobile healthcare provider user interface 1158. In an embodiment, the healthcare provider user interfaces 1157, 1158 may display information for multiple patients, wherein the information may include HRV measurements and other biomarker results, an indication of cardiovascular risk levels (e.g., different colors to indicate low vs high risk), and doctor's notes. In an embodiment, the software application may be configured to incorporate other biomarker inputs into a calculation of a cardiovascular risk score, and the software application may be configured to allow adjustment of predefined cardiovascular risk categories. The healthcare provider user interfaces 1157, 1158 may also be configured to highlight records that require immediate attention, display historical trends, and may also allow a healthcare provider to input notes that are added to a record.

It may be advantageous to set forth definitions of certain words and phrases used in this patent document. The term “couple” and its derivatives refer to any direct or indirect communication between two or more elements, whether or not those elements are in physical contact with one another. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Further, as used in this application, “plurality” means two or more. A “set” of items may include one or more of such items. Whether in the written description or the claims, the terms “comprising,” “including,” “carrying,” “having,” “containing,” “involving,” and the like are to be understood to be open-ended, i.e., to mean including but not limited to. Only the transitional phrases “consisting of” and “consisting essentially of,” respectively, are closed or semi-closed transitional phrases with respect to claims.

If present, use of ordinal terms such as “first,” “second,” “third,” etc., in the claims to modify a claim element does not by itself connote any priority, precedence or order of one claim element over another or the temporal order in which acts of a method are performed. These terms are used merely as labels to distinguish one claim element having a certain name from another element having a same name (but for use of the ordinal term) to distinguish the claim elements. As used in this application, “and/or” means that the listed items are alternatives, but the alternatives also include any combination of the listed items.

Throughout this description, the aspects, embodiments or examples shown should be considered as exemplars, rather than limitations on the apparatus or procedures disclosed or claimed. Although some of the examples may involve specific combinations of method acts or system elements, it should be understood that those acts and those elements may be combined in other ways to accomplish the same objectives.

Acts, elements and features discussed only in connection with one aspect, embodiment or example are not intended to be excluded from a similar role(s) in other aspects, embodiments or examples.

Aspects, embodiments or examples of the invention may be described as processes, which are usually depicted using a flowchart, a flow diagram, a structure diagram, or a block diagram. Although a flowchart may depict the operations as a sequential process, many of the operations can be performed in parallel or concurrently. In addition, the order of the operations may be re-arranged. With regard to flowcharts, it should be understood that additional and fewer steps may be taken, and the steps as shown may be combined or further refined to achieve the described methods.

If means-plus-function limitations are recited in the claims, the means are not intended to be limited to the means disclosed in this application for performing the recited function, but are intended to cover in scope any equivalent means, known now or later developed, for performing the recited function.

If any presented, the claims directed to a method and/or process should not be limited to the performance of their steps in the order written, and one skilled in the art can readily appreciate that the sequences may be varied and still remain within the spirit and scope of the present invention.

Although aspects, embodiments and/or examples have been illustrated and described herein, someone of ordinary skills in the art will easily detect alternate of the same and/or equivalent variations, which may be capable of achieving the same results, and which may be substituted for the aspects, embodiments and/or examples illustrated and described herein, without departing from the scope of the invention. Therefore, the scope of this application is intended to cover such alternate aspects, embodiments and/or examples. Hence, the scope of the invention is defined by the accompanying claims and their equivalents. Further, each and every claim is incorporated as further disclosure into the specification.

What is claimed is:

1. A device for determining cardiovascular risk, the device comprising:
a pulse sensor;
an LED module with at least one LED;
a wireless communication module;
a microcontroller configured to:
determine a heart rate variability (HRV) value from data recorded by the pulse sensor;
determine a cardiovascular risk score for the HRV value;
cause the LED module to indicate a cardiovascular risk category by displaying a distinct LED illumination color assigned to the cardiovascular risk category; and
cause the wireless communication module to transmit the HRV value and cardiovascular risk score to a user mobile device; and
a housing comprising:
a housing body including a cavity configured to hold the pulse sensor, LED module, wireless communication module, and microcontroller; and
a housing lid enclosing the cavity and having an opening through which the pulse sensor measures a user's pulse.

2. The device of claim 1, wherein the device is configured to record HRV values and cardiovascular risk scores to a removable data storage device.

3. The device of claim 1 further comprising a housing cover pivotally attached to the housing body, wherein the cover protects the pulse sensor.

4. The device of claim 1, wherein the cardiovascular risk score is categorized as normal, borderline, or abnormal based on the measured HRV value and on predefined HRV range categories.

5. The device of claim 1, wherein the HRV value is determined by measuring variations in time intervals between successive heartbeats over a time period of at least 60 seconds.

6. The device of claim 5, wherein the time intervals between successive heartbeats are R-R intervals between successive heartbeats.

7. A system for monitoring cardiovascular risk comprising:
a cloud server;
a patient mobile device configured to upload data to the cloud server;
a healthcare provider mobile device configured to receive data from the cloud server; and
an HRV measurement device comprising a pulse sensor, LED display, and wireless communication module, wherein the HRV measurement device is configured to:
measure a pulse of a patient;
determine an HRV value from the pulse measurement;
determine a cardiovascular risk score from the HRV value;
indicate a cardiovascular risk category by displaying a distinct LED illumination color assigned to the cardiovascular risk category;
transmit the HRV value and cardiovascular risk score wirelessly to the patient mobile device; and
determine an average HRV value from multiple HRV measurements.

8. The system of claim 7 further comprising a patient user interface accessed on the patient mobile device and a healthcare provider user interface accessed on the healthcare provider mobile device.

9. The system of claim 8, wherein the healthcare provider user interface displays HRV values and cardiovascular risk scores for multiple patients.

10. The system of claim 7, wherein HRV values and cardiovascular risk scores are automatically transmitted in real time to the healthcare provider mobile device.

11. The system of claim 7, wherein the HRV measurement device is further configured to record HRV values and cardiovascular risk scores to a removable data storage device.

12. The system of claim 7, wherein the cardiovascular risk score is categorized as normal, borderline, or abnormal based on the measured HRV value and on predefined HRV range categories.

13. The system of claim 7, wherein the HRV is determined by measuring variations in time intervals between successive heartbeats over a time period of at least 60 seconds.

14. The system of claim 13, wherein the time intervals between successive heartbeats are R-R intervals between successive heartbeats.

15. A method of monitoring cardiovascular risk comprising the steps of:
recording a pulse of a patient using a pulse sensor of an HRV measurement device;
determining an HRV value from the pulse data recorded by the pulse sensor;
determining a cardiovascular risk score from the HRV value;
indicating a cardiovascular risk category by displaying a distinct LED illumination color assigned to the cardiovascular risk category;
transmitting the HRV value and cardiovascular risk score wirelessly to a patient mobile device;
uploading the HRV value and cardiovascular risk score to a cloud server;
transmitting the HRV value and cardiovascular risk score to a healthcare provider mobile device; and
determining an average HRV value from multiple HRV measurements.

16. The method of claim 15, wherein the patient views HRV values and cardiovascular risk scores through a patient user interface accessed on the patient mobile device, and wherein a healthcare provider views HRV values and cardiovascular risk scores through a healthcare provider user interface accessed on the healthcare provider mobile device.

17. The method of claim 15, wherein the HRV values and cardiovascular risk scores are recorded to a removable data storage device.

18. The method of claim 15, wherein the cardiovascular risk score is categorized as normal, borderline, or abnormal based on the measured HRV value and on predefined HRV range categories.

19. The method of claim 15, wherein the HRV is determined by measuring variations in time intervals between successive heartbeats over a time period of at least 60 seconds.

20. The method of claim 19, wherein the time intervals between successive heartbeats are R-R intervals between successive heartbeats.
